(19) United States
(12) Patent Application Publication
(10) Pub. No.: US 2020/0181576 A1
(43) Pub. Date: Jun. 11, 2020

(54) THE USE OF MECHANICAL (ACOUSTIC/SUBSONIC) VIBRATION FOR A NOVEL PARADIGM IN REGENERATIVE MEDICINE AND HUMAN WELL BEING

(71) Applicants: James Ryan, Sengokuhara (JP); Carlo Ventura, Bologna (IT)

(72) Inventors: James Ryan, Sengokuhara (JP); Carlo Ventura, Bologna (IT)

(21) Appl. No.: 16/315,978

(22) PCT Filed: Jul. 7, 2017
(86) PCT No.: PCT/US2017/041153

§ 371 Date: Jan. 7, 2019

Related U.S. Application Data
(60) Provisional application No. 62/359,646, filed on Jul. 7, 2016.

(51) Int. Cl. C12N 5/077; G01Q 60/38; G01J 3/28

(52) U.S. Cl. CPC C12N 5/0657; G01J 2003/2826; G01J 3/2823; G01Q 60/38

(57) ABSTRACT

Methods of acquiring cellular and tissue vibrational patterning to identify signatures capable to induce pluripotency and commitment towards defined lineages, as well as survival under hostile conditions (i.e. oxidative stress) in both human adult stem cells and human adult somatic cells are described. Specifically, the invention relates to the delivery of such signatures to human adult stem cells or human adult somatic cells to induce specific differentiation processes and promote survival to hostile environmental conditions. Further methods are described of targeting tissue resident in vivo to retrieve their ability to sustain self-healing process, therefore affording a Regenerative Medicine executed without the needs for (stem) cell or tissue transplantation.

### CROSS REFERENCE TO RELATED APPLICATIONS

The present application claims priority under to U.S. Provisional Patent Application Ser. No. 62/359,646 filed Jul. 7, 2016, which is incorporated herein in its entirety.

### FIELD OF THE INVENTION

The present invention relates to acquiring cellular and tissue vibrational patterning to identify “signatures” capable to induce pluripotency and commitment towards defined lineages, as well as survival under hostile conditions (i.e. oxidative stress) in both human adult stem cells and human adult somatic cells. Specifically, the present invention provides methods to:
Deliver such signatures to human adult stem cells or human adult somatic cells to induce specific differentiation processes and promote survival to hostile environmental conditions.
Target tissue resident in vivo to retrieve their ability to sustain self-healing process, therefore affording a Regenerative Medicine executed without the needs for (stem) cell or tissue transplantation.

### BACKGROUND OF THE INVENTION

There is now compelling evidence that our cells and their subcellular structures generate and perceive mechanical oscillations (1-3).

Biomolecular recognition is also insolvably linked to the oscillatory nature of subcellular components. The conventional view is that signaling molecules have to interact like a key in a lock to trigger an event. This is certainly the case, but there is also evidence indicating that the cellular reactions exhibit a timely, wide-ranging connectedness which is too fast to be explained solely upon the simple molecular diffusion in water. Most of water molecules are bound to subcellular structures, which are constantly moving and oscillating, like the cytoskeleton and the nucleoskeleton, forming a sort of textile network, encompassing the nucleus, mitochondria and the endoplasmic reticulum, that will create serious problems to a merely diffusive trafficking of signaling molecules.

If we think of proteins in physical terms, we may see their alpha-helices as being like springs and the turns between them as connectors, making the system in a single protein capable to vibrate in a sort of phase resonance. This oscillator (the protein) is like a metronome, which owing to molecular motors like kinesins or dyneins, is able to move along the cyto-nucleo-skeleton, where the microtubules act as an elastic network dissipating the major rhythmic differences among the various oscillators that compose the ensemble of signaling molecules (4, 5). This context facilitates and promotes the achievement of synchronization phases, with each element of the network remaining individual and, at the same time becoming aware of what is occurring in the system, because of its inherent connectedness.

Consonant with this view, the cellular microtubuli, due to their intrinsic vibration modes and electrical polarity, are now regarded as a system capable of generating high-frequency electric fields with radiation characteristics (6-8). The overall oscillating field (both mechanical and electromagnetic) provided by the microtubular network appears to be important for the intracellular organization and intercellular interaction. These motions are highly coordinated, also being associated with motor proteins moving along cytoskeletal filaments, and by the dynamic growth and shrinkage of the filaments themselves.

Intra/inter cellular motility appears to be coordinated through mechanical signals passing between and regulating the activity of motors, microtubuli, and filaments. These signals are carried by forces and sensed through the acceleration of protein-protein dissociation rates. Mechanical signaling can lead to spontaneous symmetry breaking, switching, and oscillations, and it can account for a wide range of cell motions such as mitotic spindle movements, and bidirectional organelle transport and the establishment of collective behaviors, as those afforded by cell signaling networks. Because forces can propagate quickly, mechanical signaling is ideal for coordinating motion and information over large distances.

Biomolecular recognition patterning through resonance is a remarkably intriguing process, since it enables selective energy transfer with minimum energy loss. Resonances at the level of biological macromolecules, such as proteins, DNA and RNA, are particularly rewarding, as they relate to and can impact with the biological activity of the macromolecule itself. Distinct vibrational modes of macromolecules in the high frequency domain stem from their intra-molecular degree of freedom, with every single molecule displaying a specific vibrational signature (9-11).

There is now compelling evidence for the existence of high frequency electromagnetic and mechanical oscillations of proteins, indicating that such oscillations can share a common megahertz (MHz) frequency domain (11-13). In proteins, depending on the vibrational mode triggered by an external electromagnetic frequency, the relaxation time could change from fifty nanoseconds (10⁹ Hz˜GHz) to a few hundred microseconds (10⁶ Hz˜MHz). Nevertheless, physical protein folding can vary between few microseconds and few seconds. For some proteins the electromagnetic and mechanical oscillations may have a common time or frequency region, where both electromagnetic and mechanical oscillations merge. Assuming a protein (molecule) remodeling time within a microsecond frame, the related frequency (the inverse of microseconds) will be in the megahertz range, which will identify the “common frequency point”. The merging of electromagnetic and mechanical oscillations at this “point” has the crucial implication that we can manipulate one with another.

We have shown that embryonic and human adult stem cells can be committed along specific differentiation routes by asymmetrically conveyed electromagnetic fields (ACEMF) (14-15), and that extremely-low frequency magnetic field can remarkably affect gene transcription in rat adult cardiomyocytes (16) and promote high-efficiency cardiogenesis in mouse embryonic stem cells (17). ACEMF can even be used to direct non-stem human somatic cells, like skin fibroblasts into complex lineages (i.e. cardiac, neural, skeletal muscle) in which they would never otherwise appear (18). Our recent studies have also shown that ACEMF can reverse human stem cell senescence in vitro by priming both telomerase dependent—and—independent pathways (19), and reprogram tumor cells of neural origin into dopaminergic neurons (20). Most of these effects resulted from the ability of ACEMF to optimize cell polarity (21), a crucial trait in the physiological modulation of stem cell differentiation and aging, as shown by the fact that altered cell polarization invariantly associates with disease, pathological aging and cancer (22-25). To this end, the cytoskeletal and nucleoskeletal microtubular network form a major dynamic environment to establish and preserve cell polarity. The DNA itself, considered as an electrically charged vibrational entity, despite its role of storage and expression of genetic information, may conceivably contribute to cell polarity, also by virtue of its continuous epigenetic remodeling, and architectural assembly in multifaceted loops and domains that are essential features of the nano-mechanics and nano-topography imparted to this macromolecule by the timely intervention of transcription factors and molecular motors.

Consonant with these considerations, cells have been found to generate defined vibrational patterns, and Atomic Force Microscopy (AFM) has been extensively used to monitor these nanomechanical motions across a wide spectrum of biological conditions, including the assessment of metabolic states (26), the analysis of differentiating mechanisms (27), the identification of signatures from cancer/metastatic cells (28, 29), the dissection of nanotopografic features at the level of subcellular elements, including the microtubuli (30), the actin filaments (31), and the exosome nanovesicles (32, 33).

Overall, while it is possible to affect cell biology with electromagnetic fields of different characteristics, there is compelling evidence that cells generate defined mechanical vibration and that mechanical vibration itself can trigger and modulate electromagnetic patterns in cells.

Therefore methods are needed to characterize vibrational patterning in cells and tissues associated with their health status or the attainment of defined fates or the capability to survive to hostile environment, or the possibility to reprogram diseased cells (i.e. cancer stem cells) into healthy, functionally active cells.

### SUMMARY OF THE INVENTION

The present invention provides methods to acquire specific vibrational patterns (signatures) from cells/stem cells undergoing commitment into different lineages or terminal differentiation into specific phenotypes.

In one embodiment of the present invention methods are provided to retrieve signatures for the acquirement of a pluripotent state in human adult or embryonic stem cells, as well as in human induced pluripotent stem cells (iPS) and human adult somatic cells.

Human adult stem cells can reverse their aging process due to prolonged culture in vitro when exposed to asymmetrically conveyed radio electric fields (19) or early stage developmental factors obtained from the zebrafish embryo (34). In another embodiment methods are provided to acquire vibrational patterns expressed by human adult stem cells or human somatic cells during their aging reversal.

In another embodiment defined vibrational signatures are acquired from cells/stem cells surviving oxidative stress, as induced by 1-hour exposure to H₂O₂ to generate reactive oxygen species, or 5HD (mitoKATP channel blocker) or rotenone (it works by interfering with electron transport chain in mitochondria).

In another embodiment vibrational patterns will be acquired from the human heart sound, including the identification of autosimilarity/fractal frequency patterns.

In another embodiment we show that defined vibrational patterns from the human heart sound can be applied to human adult stem cells to induce an efficient program of cardiogenesis.

In another embodiment vibrational patterns from the human heart sound can be delivered to human iPS to induce an efficient program of cardiogenesis.

In all living species, including the drosophila, rodents, amphibians, primates and Humans, cardiogenesis is the first morphogenetic event occurring in the developing embryo. This suggests that the human heart sound may store information for the attainment of other complex morphogenetic pathways than cardiogenesis itself.

In another embodiment, we show that the same vibrational signatures from the human heart that induce cardiogenesis in human adult stem cells can also be delivered to human adult stem cells to induce other complex lineages, including neurogenesis and vasculogenesis.

In another embodiment the same vibrational signatures from the human heart that induce cardiogenesis in human iPS can also be delivered to human iPS to induce other complex lineages, including neurogenesis and vasculogenesis.

In another embodiment the same vibrational signatures from the human heart that induce cardiogenesis in human adult stem cells or human iPS can be applied to human adult somatic cells to promote a multilineage commitment.

In another embodiment a method is provided to obtain specific vibrational signatures and audible sound patterns from human iPS-derived cardiomyocytes.

In another embodiment the same vibrational signatures from human iPS-derived cardiomyocytes are applied to human iPS to transform them into beating human cardiomyocytes.

In another embodiment the same vibrational signatures from human iPS-derived cardiomyocytes are applied to human iPS to transform them into neurons and endothelial cells.

In another embodiment the same vibrational signatures from human iPS-derived cardiomyocytes are applied to human adult stem cells to transform them into cardiac, neural and vascular cells.

In another embodiment the same vibrational signatures from human iPS-derived cardiomyocytes are applied in vitro to transform human adult somatic cells into cardiac, neural and endothelial cells.

In another embodiment vibrational signatures obtained as reported in [0015] are used to reverse senescence in human adult stem cells in vitro.

In another embodiment vibrational patterns acquired from cells/stem cells surviving oxidative stress, as reported in [0016], or other hostile conditions (i.e. hypoxia), are delivered to human adult stem cells or human adult somatic cells remarkably enhancing their survival. Only a small percentage (about 5%) of cells survives the oxidative stress induced as reported in [0016]. When human adult stem cells or somatic cells are first exposed for 24 hours under normal conditions to the vibrational patterns ensued from the few cells surviving the hostile conditions of the oxidative stress or hypoxia and then subjected for 1 hour to oxidative stress, or hypoxia, the percentage of surviving stem cells is dramatically enhanced, between 20 and 30% of the original population.

In another embodiment human cancer stem cells are reprogrammed in vitro by vibrational patterns acquired from the human heart sound or human iPS-derived cardiomyocyutes into elements capable of lineage commitment decisions (i.e. cardiac-, neural-, and skeletal muscle-like cells).

In another embodiment human cancer stem cells subjected in vitro to vibrational patterns acquired from the human heart sound or human iPS-derived cardiomyocyutes are remarkably commitment to apoptosis.

The achievements reported in [0028-0029] have remarkable biomedical implication. Cancer stem cells, a small number of cells within the tumor, are resistant to conventional chemotherapy and radiotherapy (35-38), and play a crucial role in the maintenance of tumor growth and initiation of metastatic process (36, 37). A new era may emerge in case cancer stem cells can gain differentiating abilities. The analysis of vibrational signatures in normal and cancer stem cells may reveal novel cues on the way these cells organize their fate. Consonant with such perspectives are compelling data showing that (i) tumours display unique mechanical properties, being considerably stiffer than normal tissue (28, 29) and that (ii) the mechanical microenvironment may cause malignant transformation (39). Hence, the application of localized forces, the use of localized probes, nanopatterned substrates or substrates designed to apply localized forces, may eventually become a strategy to enhance or direct cellular differentiation in cancer stem cells. Why vibrational signatures form the human heart sound and from human iPS-derived cardiomyocytes can funnel cancer stem cells into differentiating and apoptotic decisions? Intriguingly, the heart has the lowest risk for primary malignant transformation, which may very rarely develop in the form of cardiac sarcomas (40-42). Cardiogenesis is the first morphogenetic event in different animal species, including humans. The risk for tumorigenesis throughout embryo development is also very rare (43-45). The canonical view speculating that primary cardiac malignant tumors are so rare since cardiac cells divide very rarely appears to be too simplistic. An alternative although non-mutually exclusive hypothesis may consider the heart as a tumor suppressor organ, capable of secreting a large network of growth regulatory and differentiating peptides that may potentially limit the onset and progression of a local cancer. In this regard, we have shown that the attainment of cardiogenesis in the presence of either chemical agents or physical stimulation encompasses the transcription and protein expression of endorphin peptides (46). These molecules, besides their role in cardiogenesis (47-49), have long been shown to act as negative regulators for the development and spreading of different types of cancer (50-57). Mechanical signatures for cardiogenesis, acquired either from the sound of the human adult heart or through the analysis of mechanical vibrations appearing during the cardiogenesis of human iPS can be used to control cancer stem cells dynamics. When exposed to cardiogenic vibrational patterning, cancer stem cells will be committed to differentiate into phenotypes of their tissue of origin, or conversely will enter apoptotic pathways. This approach will place the cancer therapy within the context of Regenerative Medicine.

In another embodiment vibrational patterns acquired from the human heart sound are used to modulate the content and release from exosome nanovesicles, therefore controlling the intercellular trafficking of building blocks of information.

In another embodiment vibrational patterns acquired from human iPS-cardiomyocytes are used to modulate the content and release from exosome nanovesicles, therefore providing an additional example for the capability of vibrational patterns to control intercellular communication.

In another embodiment of the invention vibrational patterns are delivered in vivo to any part of the human body, or animal body in the case of veterinary use, with the specific aim of targeting and reprogramming stem cells where they are, in all tissues. As a result, the invention aims at deploying the diffusive features of vibrational mechanical forces to afford a Regenerative Medicine based upon retrieving the natural self-healing capabilities ensuing from optimization of cellular polarity and differentiating/paracrine dynamics from tissue resident stem tells, without the needs for stem cell transplantation.

### DETAILED DESCRIPTION OF THE INVENTION

### Systems Used for Detection of Vibrational Patterns in Cells or Tissues

Atomic Force Microscopy (AFM).

The AFM is a scanning probe microscope that measures a local property, such as topography, mechanical properties, thermal and electrical properties, optical absorption or magnetism, with a probe or “tip” placed very close to the sample. The small probe-sample separation makes it possible to take measurements over a small area. Because the AFM can image biological samples at sub-nanometer resolution in their natural aqueous environment, it has potential for characterization of living cells. Using the AFM, it has been possible to observe living cells under physiologic conditions, detecting and applying small forces with high sensitivity (26). In yeast and bacterial cells, cellular activity, metabolism, growth and morphogentic changes were associated with defined nanomechanical activity, merging to the cell surface up to the generation of defined patterns of vibrations (26). “Sonocytology” is the term that has been introduced to identify a novel area of inquiry based on the fact that in these small cells, after an accurate process of amplification, given the frequency range of nanomechanical motions recorded by AFM, the vibrations could be transformed into audible sounds, providing a thorough assessment of mechanistic cellular dynamics (26). More complex eukaryotic cells can also be investigated by this approach. For example, stem cells directed to cardiac myocyte differentiation begin to beat at a point in differentiation. This beating motion requires a major reorganization of the cell cytoskeleton and microtubuli, and in turn a significant change in cellular nanomechanical properties. Concerning the cytoskeleton, it is now evident that transferring of mechanical vibration to the subcellular environment triggers the mobilization of ionic species and the generation of ionic fluxes and induced microcurrents, ultimately ensuing in the appearance of oscillating electromagnetic fields (6-8). Considering the remarkable biological effects of electromagnetic fields, and their recently reported ability to control stem cell dynamics, including pluripotency, differentiation and senescence, the modulation of cellular electromagnetic patterning may represent an additional level of cell regulation afforded through the application of mechanical forces.

Hyper Spectral imaging (HSI).

HSI, also known as imaging spectrometer, relies upon the advantage of acquiring two-dimensional images across a wide range of electromagnetic spectrum. HSI is now subjected to multiple applications in wide-ranging contexts, including archaeology and art conservation, food quality and safety control, and biomedicine (for a recent comprehensive review, see Ref. 58). HSI can be regarded as an emerging imaging tool with remarkable potential for non-invasive biomedical diagnosis and assessment. Light delivered to biological tissues in vivo undergoes multiple scattering from inhomogeneity of biological structures and absorption primarily in signaling molecules and water as it propagates through the tissues (58, 59). HSI can provide nearly real-time images from informational biomarkers, including oxyhemoglobin and deoxyhemoglobin, affording an estimation of tissue homeostasis based upon molecular spectral characteristics within various tissues (58-60). Relevant to this invention HSI is now recognized as a major tool to afford nanoscale vibrational imaging in living cells, providing an unprecedented platform for Biology and Medicine (61, 62). HSI has also been shown to provide unprecedented cues of the features of differentiating stem cells at both quantitative and qualitative levels in a non-invasive fashion (63). Within this context, HSI will be exploited to analyze subtle vibrational modes from the initial period of stem cell commitment to cardiogenesis, when sporadic non-coherent wave forms of contraction begin up to the development of coherent vibrational modes underlying the appearance of synchronous beating (twitch).

In the present invention both. AFM and HSI are used to obtain vibrational signatures from cells/stem cells under the above reported conditions. In fact, AFM provides a thorough estimation of nanomechanical motions and their underlying force development in space and time. HSI provides measurement of the electromagnetic radiation reflected from an object or scene (i.e., materials in the image) at many narrow wavelength bands. By the aid of a multispectral camera adapted to the stage of an inverted microscope we use a dedicated software for “floating point” analyses of pixel reflection at all given wavelengths. This analysis yields spatial resolution of fluctuations in pixel luminance (i.e. the intensity of light emitted from a surface per unit area in a given direction) and chrominance (i.e. the colorimetric difference between a given color in a picture and a standard color of equal luminance), corresponding to a pixel-related spectral signature. In this regard, HSI may offer several advantages over AFM for the recording of the vibrational pattern of cells, as HSI is not affected by the bias introduced by the contact modes of the AFM cantilever tip with the cell surface, which may itself suppress weaker nanomotions, erasing relevant vibrational information.

### Systems Used to Deliver Defined Vibrational Patterns to Cells or Tissue In Vivo

In another embodiment we provide vibrational actuators working as devices for stem cell or somatic cell reprogramming in vitro.

In another embodiment we provide vibrational devices forged to interact and adapt in vivo with any part of the body in order to target the underlying tissue-resident stem cell population(s).

In another embodiment we provide vibrational devices suitable for being embedded in smart phones, pad, tablets.

In another embodiment we provide vibrational actuators forged for being embedded within textile structures, becoming part of vests/dressing bearing vibrational codes for the self-healing/rescue of damaged tissues.

In another embodiment we provide armchairs capable to deliver defined vibrational patterning to the spine and use it as a second actuator for the spreading of vibration to other parts of the body.

In another embodiment we provide Pods capable to embed the whole body in a dedicated vibrational environment.

In another embodiment we provide vibrational actuators embedding graphene nanolayers or carbon nanotubes. These actuators not only will be able to deliver specific vibrational signatures to all parts of the human body, but they will merge the delivery of vibrational patterns with the unique optical and electrical properties of graphene or carbon nanotubes that can stimulate and further assist stem cell differentiation. So far, pulse electrical stimulation has been shown to enhance neuronal regeneration and graphene-based powered electrical stimulation has been shown to remarkably enhance stein cell neurogenesis (64, 65). Graphene substrate can act as a conductive substrate, interacting with and optimizing the cellular/tissue microcurrents generated by the mechanical motions applied with vibrational actuators. Within this combined action, graphene may act as a sender-and-receiver of cellular/tissue microcurrents optimizing cell polarity and the related mobility at the level of cytoskeleton and nucleoskeleton.

The generation of vibrational actuators embedding graphene/carbon nanotubes results in higher efficiency of stem cell differentiation and secretion of rescuing/regenerative factors.

The diffusive nature of physical vibrational forces delivered alone or in combination with the optical and electrical properties of graphene or carbon nanotubes will allow targeting of tissue-resident stem cells in all tissues in vivo.

Translation of electrical signals into vibrational patterns is afforded by the aid of ad-hoc designed mechanical transducers and signal generators for maximal fidelity delivery between 5 and 20000 Hz.

The application of vibrational forces with defined signatures in vivo alone or in combination with the optical and electrical properties of graphene or carbon nanotubes will optimize the differentiating repertoire, the paracrine patterns from exosomal routes, and it will reverse aging process in tissue-resident stem cells, enhancing the self-healing potential where it's mostly needed, at the level of damaged/deranged tissues.

In another embodiment we provide scalable Textile Artworks behaving as multisensory dynamically interactive environments targeted to promote human well-being.

Textile Sculptures are fashioned to dynamically interact with a single subject (explorer) or multiple explorers at the same time, in order to sense and deploy human heart and brain waves into vibrational symphonies that will be fed back to the explorers to amplify their multisensory repertoire and provide unprecedented “perceptions” for well-being and self healing paths.

Multisensorial domes are created to transform perceptions from heart and brain waves into a novel form of synchronization: The Untold Prayer, to create coherence with immaterial and spiritual dimensions.

### REFERENCES: DETAILED DESCRIPTION OF THE INVENTION

1. Albrecht-Buehler G. Rudimentary form of cellular “vision”. Proc Natl Acad Sci USA 1992; 89:8288-8292.
2. Albrecht-Buehler G. A long-range attraction between aggregating 3T3 cells mediated by near-infrared light scattering. Proc Natl Acad Sci USA 2005; 102:5050-5055.
3. Uzer G, Thompson W R, Sen B, Xie Z, Yen S S, Miller S, Bas G, Styner M, Rubin C T, Judex S, Burridge K, Rubin J. Cell Mechanosensitivity to Extremely Low-Magnitude Signals Is Enabled by a LINCed Nucleus. Stem cells 2015; 33:2063-2076.
4. Martens E A, Thutupalli S, Fourriere A, Hallatschek O. Chimera states in mechanical oscillator networks. Proc Natl Acad Sci USA 2013; 110:10563-10567.
5. Schaap I A, Carrasco C, de Pablo P J, Schmidt C F. Kinesin walks the line: single motors observed by atomic force microscopy. Biophys J 2011; 100:2450-2456.
6. Havelka D, Cifra M, Kueera O, Pokorný J, Vrba J. High-frequency electric field and radiation characteristics of cellular microtubule network. J Theor Biol 2011; 286:31-40.
7. Sahu S, Ghosh S, Hirata K, Fujita D, Bandyopadhyay A. Multi-level memory-switching properties of a single brain microtubule. Appl Phys Lett 2013; 102:123701. doi:
8. Sahu S, Ghosh S, Fujita D, Bandyopadhyay A. Live visualizations of single isolated tubulin protein self-assembly via tunneling current: effect of electromagnetic pumping during spontaneous growth of microtubule. Sci Rep 2014 Dec. 3; 4:7303. doi: 10.1038/srep07303.
9. Doster W, Diehl. M, Leyser H, Petry W, Schober H. [Terahertz spectroscopy of proteins: Viscoelastic damping of boson peak oscillations.] Spectroscopy of Biological Molecules: New Directions [Greve, J., Puppels, G. J., Otto C. (Eds.)] [655-658] (Springer publications, Netherlands, 1999),
10. Upadhya P C, et al. Characterization of Crystalline Phase Transformations in Theophylline by Time-Domain Terahertz Spectroscopy. Spect Lett 2006; 39:215-224.
11. Gilmanshin R, Williams S, Callender R H, Woodruff W H, Dyer R. B. Fast events in protein folding: Relaxation dynamics of secondary and tertiary structure in native apomyoglobin. Proc Natl Acad Sci USA 1997; 94:3709-3713.
12. Aronsson G, Brorsson A C, Sahlman L, Jonsson B H. Remarkably slow folding of a small protein. FEBS Lett 1997; 411:359-364.
13. Mithieux G, Chauvin F, Roux B, Rousset B. Association states of tubulin in the presence and absence of microtubule-associated proteins. Analysis by electric birefringence. Biophys Chem 1985; 22:307-316.
14. Maioli M, Rinaldi S, Santaniello S, Castagna A, Pigliaru G, Gualini S, Fontani V, Ventura C. Radio frequency energy loop primes cardiac, neuronal, and skeletal muscle differentiation in mouse embryonic stem cells: a new tool for improving tissue regeneration. Cell Transplant 2012; 21:1225-1233.
15. Maioli M, Rinaldi S, Santaniello S, Castagna A, Pigliaru G, Delitala A, Bianchi F, Tremolada C, Fontani V, Ventura C. Radio electric asymmetric conveyed fields and human adipose-derived stem cells obtained with a non-enzymatic method and device: a novel approach to multipotency. Cell Transplant 2014; 23:1489-1500.
16. Ventura C, Maioli M, Pintus G, Gottardi G, Bersani F. Elf-pulsed magnetic fields modulate opioid peptide gene expression in myocardial cells. Cardiovasc Res 2000; 45:1054-1064.
17. Ventura C, Maioli M, Asara Y, Santoni D, Mesirca P, Remondini D, Bersani F. Turning on stem cell cardiogenesis with extremely low frequency magnetic fields. FASEB J 2005; 19:155-157.
18. Maioli M, Rinaldi S, Santaniello S, Castagna A, Pigliaru G, Gualini S, Cavallini C, Fontani V, Ventura C. Radio electric conveyed fields directly reprogram human dermal-skin fibroblasts toward cardiac-, neuronal-, and skeletal muscle-like lineages. Cell Transplant 2013; 22:1227-1235.
19. Rinaldi S, Maioli M, Pigliaru G, Castagna A, Santaniello S, Basoli V, Fontani V, Ventura C. Stem cell senescence. Effects of REAC technology on telomerase-independent and telomerase-dependent pathways. Sci Rep 2014 Sep. 16; 4:6373. doi: 10.1038/srep06373.
20. Maioli M, Rinaldi S, Migheli R, Pigliaru G, Rocchitta G, Santaniello S, Basoli V, Castagna A, Fontani V, Ventura C, Serra P A. Neurological morphofunctional differentiation induced by REAC technology in PC12. A neuro protective model for Parkinson's disease. Sci Rep 2015 May 15; 5:10439. doi: 10.1038/srep10439.
21. Maioli M, Rinaldi S, Pigliaru G, Santaniello S, Basoli V, Castagna A, Fontani V, Ventura C. REAC technology and hyaluron synthase 2, an interesting network to slow down stem cell senescence. Sci Rep 2016 Jun. 24; 6:28682. doi: 10.1038/srep28682.
22. Florian M C, Geiger H. Concise review: polarity in stem cells, disease, and aging. Stem Cells 2010; 28:1623-1629.
23. Lee M, Vasioukhin V. Cell polarity and cancer—cell and tissue polarity as a non-canonical tumor suppressor. J Cell Sci 2008; 121(Pt 8):1141-1150.
24. Wodarz A, Näthke I. Cell polarity in development and cancer. Nat Cell Biol 2007; 9(9):1016-1024.
25. Martin-Belmonte F, Perez-Moreno M. Epithelial cell polarity, stem cells and cancer. Nat Rev Cancer 2011; 12(1):23-38.
26. Pelling A E, Sehati S, Gralla E B, Valentine J S, Gimzewski J K. Local nanomechanical motion of the cell wall of Saccharomyces cerevisiae. Science 2004; 305:1147-1150.
27. Arshi A, Nakashima Y, Nakano H, Eaimkhong S, Evseenko D, Reed J, Stieg A Z, Gimzewski J K, Nakano A. Rigid microenvironments promote cardiac differentiation of mouse and human embryonic stem cells. Sci Technol Adv Mater. 2013 Aug. 1; 14(2). pii: 025003.
28. Cross S E, Jin Y S, Rao J, Gimzewski J K. Nanomechanical analysis of cells from cancer patients. Nat Nanotechnol. 2007 December; 2(12):780-3. doi: 10.1038/nnano.2007.388. Epub 2007 Dec. 2.
29. Cross S E, Jin Y S, Tondre J, Wong R, Rao S, Gimzewski J K. AFM-based analysis of human metastatic cancer cells. Nanotechnology. 2008 Sep. 24; 19(38):384003. doi: 10.1088/0957-4484/19/38/384003. Epub 2008 Aug. 12.
30. Pelling A E, Dawson D W, Carreon D M, Christiansen J J, Shen R R, Teitell M A, Gimzewski J K. Distinct contributions of microtubule subtypes to cell membrane shape and stability. Nanomedicine. 2007 March; 3(1):43-52.
31. Sharma. S, Grintsevich E E, Hsueh C, Reisler E, Gimzewski J K. Molecular cooperativity of drebrin1-300 binding and structural remodeling of F-actin. Biophys J. 2012 Jul. 18; 103(2):275-83. doi: 10.1016/j.bpj.2012.06.006. Epub 2012 Jul. 17.
32. Sharma S, Gillespie B M, Palanisamy V, Gimzewski J K. Quantitative nanostructural and single-molecule force spectroscopy biomolecular analysis of human-saliva-derived exosomes. Langmuir. 2011 Dec. 6; 27(23):14394-400. doi: 10.1021/1a2038763. Epub 2011 Nov. 9.
33. Sharma S, Rasool H I, Palanisamy V, Mathisen C, Schmidt M, Wong D T, Gimzewski J K. Structural-mechanical characterization of nanoparticle exosomes in human saliva, using correlative AFM, FESEM, and force spectroscopy. ACS Nano. 2010 Apr. 27; 4(4):1921-6. doi: 10.1021/nn901824n.
34. Canaider S, Maioli M, Facchin, F, Bianconi E, Santaniello S, Pigliaru G, Ljungberg L, Burigana F, Bianchi F, Olivi E, Tremolada C, Biava P M, Ventura C. Human Stem Cell Exposure to Developmental Stage Zebrafish Extracts: a Novel Strategy for Tuning Sternness and Senescence Patterning. Cell R4, 2014; 2(5), e1226.
35. Coleman W B, Wennerberg A E, Smith G J, Grisham J W. Regulation of the differentiation of diploid and some aneuploid rat liver epithelial (stemlike) cells by the hepatic microenvironment. Am J Pathol 1993; 142(5):1373-1382.
36. Visvader J E, Lindeman G J. Cancer stem cells: current status and evolving complexities. Cell Stem Cell 2012; 10(6):717-728. doi: 10.1016/j.stem.2012.05.007.
37. Reya T, Morrison S J, Clarke M F, Weissman I L. Stein cells, cancer and cancer stem cells. Nature 2001; 414(6859):105-111.
38. Shackleton M, Quintana E, Fearon E R, Morrison S J. Heterogeneity in cancer: cancer stem cells versus clonal evolution. Cell 2009; 138(5):822-829. doi: 10.1016/j.cell.2009.08.017.
39. Nagelkerke A, Bussink J, Rowan A E, Span P N. The mechanical microenvironment in cancer: How physics affect tumours. Semin Cancer Biol. 2015; 35:62-70. doi: 10.1016/j.semcancer.2015.09.001.
40. Goldberg H P, Steinberg I. Primary tumors of the heart. Circulation 1955; 11(6):963-970.
41. Leja M J, Shah D J, Reardon M J. Primary cardiac tumors. Tex Heart Inst J. 2011; 38(3):261-262.
42. Hudzik B, Miszalski-Jamka K, Glowacki J, Lekston A, Gierlotka M, Zembala M, Polonski L, Gasior M. Malignant tumors of the heart. Cancer Epidemiol. 2015; Jul. 31. pii: S 1877-7821(15)00159-9. doi: 10.1016/j.canep.2015.07.007.
43. Pierce G B. The cancer cell and its control by the embryo. Am J Pathol 1983; 113 (1):117-124.
44. Hendrix M J, Seftor E A, Seftor R E B, Kaisermeier-Kulesa J, Kulesa P M, Postovit L M. Reprogramming metastatic tumor cells with embryonic microenvironment. Nat Rev Cancer 2007; 7(4):246-255.
45. Postovit L M, Maragaryan N V, Seftor E A, Kirschmann D A, Lipayski A, Wheaton W W, Abbott D E, Seftor R E, Hendrix M J. Human embryonic stem cell microenvironment suppress the tumorigenic phenotype of aggressive cancer cells. Proc Natl Acad Sci USA 2008; 105(11): 4329-34. doi: 10.1073/pnas.0800467105.
46. Ventura C, Maioli M, Asara Y, Santoni D, Scarlata I, Cantoni S, Perbellini. A. Butyric and retinoic mixed ester of hyaluronan: a novel differentiating glycoconjugate affording a high-throughput of cardiogenesis in embryonic stem cells. J Biol Chem 2004; 279(22): 23574-23579.
47. Ventura C, Zinellu E, Maninchedda E, Fadda M, Maioli M. Protein kinase C signaling transduces endorphin-primed cardiogenesis in GTRI embryonic stem cells. Circ Res 2003; 92(6):617-622.
48. Ventura C, Zinellu E, Maninchedda E, Maioli M. Dynorphin B is an agonist of nuclear opioid receptors coupling nuclear protein kinase C activation to the transcription of cardiogenic genes in GTRI embryonic stem cells. Circ Res 2003; 92(6):623-629.
49. Ventura C, Maioli M. Opioid peptide gene expression primes cardiogenesis in embryonal pluripotent stem cells. Circ Res 2000; 87(3):189-194.
50. Melander O, Orho-Melander M, Manjer J, Svensson T, Almgren P, Nilsson P M, Engstrom G, Hedblad B, Borgquist S, Hartmann O, Struck J, Bergmann A, Belting M. Stable Peptide of the Endogenous Opioid Enkephalin Precursor and Breast Cancer Risk. J Clin Oncol 2015; 33(24): 2632-2638. doi: 10.1200/JCO.2014.59.7682.
51. Banerjee J, Papu John A M, Schuller H M. Regulation of nonsmall-cell lung cancer stem cell like cells by neurotransmitters and opioid peptides. Int J Cancer. 2015; Jun. 18. doi: 10.1002/ijc.29646. [Epub ahead of print].
52. McLaughlin P J, Zagon I S. Novel treatment for triple-negative breast and ovarian cancer: endogenous opioid suppression of women's cancers. Expert Rev Anticancer Ther 2014; 14(3): 247-250. doi: 10.1586/14737140.2014.867234.
53. Sarkar D K, Murugan S, Zhang C, Boyadjieva N. Regulation of cancer progression by β-endorphin neuron. Cancer Res 2012; 72(4):836-840. doi: 10.1158/0008-5472.CAN-11-3292.
54. Melander O, Orho-Melander M, Manjer J, Svensson T, Almgren P, Nilsson P M, Engström G, Hedblad B, Borgquist S, Hartmann O, Struck J, Bergmann A, Belting M. Stable Peptide of the Endogenous Opioid Enkephalin Precursor and Breast Cancer Risk. J Clin Oncol 2015; 33(24):2632-2638. doi: 10.1200/JCO.2014.59.7682.
55. Banerjee J, Papu John A M, Schuller H. M. Regulation of nonsmall-cell lung cancer stem cell like cells by neurotransmitters and opioid peptides. Int J Cancer 2015; Jun. 18. doi: 10.1002/ijc.29646. [Epub ahead of print].
56. Wang Q, Gao X, Yuan Z, Wang Z, Meng Y, Cao Y, Plotnikoff N P, Griffin N, Shan F. Methionine enkephalin (MENK) improves lymphocyte subpopulations in human peripheral blood of 50 cancer patients by inhibiting regulatory T cells (Tregs). Hum Vaccin Immunother. 2014; 10(7):1836-1840. doi: 10.4161/hv.28804.
57. Zagon I S, McLaughlin P J. Opioid growth factor and the treatment of human pancreatic cancer: a review. World J Gastroenterol 2014; 20(9):2218-23. doi: 10.3748/wjg.v20.i9.2218.
58. Guolan Lu, Baowei Fei. Medical hyperspectral imaging: a review. J Biomed Opt 2014 January; 19(1): 010901. Published online 2014 Jan. 20. doi: 10.1117/JBO.19.1.010901
59. Gao L, Smith R T. Optical hyperspectral imaging in microscopy and spectroscopy—a review of data acquisition. J Biophotonics 2014 Sep. 3; 9999(9999). doi: 10.1002/jbio.201400051.
60. Lu G. Halig L, Wang D, Qin X, Chen Z G, Fei B. Spectral-spatial classification for noninvasive cancer detection using hyperspectral imaging. J Biomed Opt 2014:19(10):106004, doi: 10.1117/JBO.19.10.106004.
61. Schultz R A, Nielsen T, Zavaleta J R, Ruch R, Wyatt R, Garner H R. Hyperspectral imaging: a novel approach for microscopic analysis. Cytometry. 2001; 43(4):239-247.
62. Cheng J X, Xie X S. Vibrational spectroscopic imaging of living systems: An emerging platform for biology and medicine. Science 2015 Nov. 27; 350(6264):aaa8870. doi: 10.1126/science.aaa8870.
63. Gosnell M E, Anwer A G, Mahbub S B, Menon Perinchery S, Inglis D W, Adhikary P PJazayeri J A, Cahill M A, Saad S, Pollock C A, Sutton-McDowall M L, Thompson J G, Goldys E M. Quantitative non-invasive cell characterisation and discrimination based on multispectral autofluorescence features. Nature Sci Rep 2016 Mar. 31; 6:23453. doi: 10.1038/srep23453.
64. Guo W, Zhang X, Yu X, Wang S, Qiu J, Tang W, Li L, Liu H, Wang Z L. Self-powered electrical stimulation for enhancing neural differentiation of mesenchymal stem cells on graphene-poly(3,4-ethylenedioxythiophene) hybrid microfibers. ACS Nano 2016; 10:5086-5095.
65. Akhavan O, Ghaderi. E. The use of graphene in the self-organized differentiation of human neural stem cells into neurons under pulsed laser stimulation. J Mater Chem B 2014; 2: 5602-5611.

1. A method to acquire specific vibrational signatures for pluripotency from human stem cells, iPS and somatic cells.

2. A method to acquire specific vibrational signatures for defined (stem) cell commitment and terminal differentiation.

3. A method to acquire vibrational signatures for aging reversal in stem cells and somatic cells.

4-24. (canceled)
